(19) United States
(12) Patent Application Publication
(10) Pub. No.: US 2003/0065344 A1
(43) Pub. Date: Apr. 3, 2003

(54) Method and device for creating microvascular anastomoses

(76) Inventors: Wolff M. Kirsch, Redlands, CA (US); Cindy Dickson, Mentone, CA (US); Yong H. Zhu, Redlands, CA (US)

Correspondence Address: SHELDON & MAK, INC, 225 SOUTH LAKE AVENUE, 9TH FLOOR, PASADENA, CA, 91101, US

(21) Appl. No.: 10/076,889

(22) Filed: Feb. 13, 2002

Related U.S. Application Data
(60) Provisional application No. 60/347,108, filed on Jan. 8, 2002.
(60) Provisional application No. 60/340,214, filed on Dec. 14, 2001.
(60) Provisional application No. 60/327,196, filed on Oct. 3, 2001.

(51) Int. Cl. A61B 17/08

(52) U.S. Cl. 606/153

(57) ABSTRACT

A coupler or a coupling vessel for creating an anastomosis between a first vessel and a second vessel, the coupler comprising a proximal portion comprising a tubular portion having a proximal end, a distal end, an outer surface, an inner surface, and an axial lumen defined by the inner surface and extending between the proximal end and the distal end, and a distal portion comprising a flange surrounding the distal end of the tubular portion. A method for creating an anastomosis between a first vessel and a second vessel, the method comprising selecting the first vessel and the second vessel, providing a coupler or a coupling vessel according to the present invention, and joining the first vessel to the coupler through the proximal end of the tubular portion and joining the second vessel to the coupler through the distal portion, thereby creating an anastomosis with fluid continuity between the first vessel and the second vessel.

### CROSS-REFERENCE TO RELATED APPLICATION

The present Application claims the benefit of U.S. provisional patent application No. 60/347,108, filed Jan. 8, 2002, entitled “Method and Devices for Constructing Microvascular Anastomoses;” and claims the benefit of U.S. provisional patent application No. 60/340,214, filed Dec. 14, 2001, entitled “Method and Devices for Constructing Microvascular Anastomoses;” and claims the benefit of U.S. provisional patent application No. 60/327,196, filed Oct. 3, 2001, entitled “Method and Devices for Constructing Microvascular Anastomoses,” the contents of which are incorporated in this disclosure by reference in their entirety.

### BACKGROUND

There are a variety of diseases and conditions affecting the distal branches of the arterial system and other hollow tubular structures having small diameters in the human body.

One epidemiologically important set of diseases involve narrowing or occlusion in the distal coronary arteries leading to cardiac ischemia. Present surgical treatment of these diseases includes the placement of grafts to bypass the narrowing or occlusion. However, the placement of these grafts is partly limited by the inability to suture grafts to distal coronary arteries of very small diameter.

Therefore, it would be useful to have a method of joining one hollow tubular vessel to another hollow tubular vessel to place the two vessels in fluid communication with each other, where at least one of the hollow tubular vessels has a very small diameter, such as a coronary artery. Further, it would be useful to have a device for joining one hollow tubular vessel to another hollow tubular vessel to place the two vessels in fluid communication with each other, where at least one of the hollow tubular vessels has a very small diameter, such as a coronary artery.

### SUMMARY

According to one embodiment, the present invention is a coupler for creating an anastomosis between a first vessel and a second vessel. The coupler comprises a proximal portion comprising a tubular portion having a proximal end, a distal end, an outer surface, an inner surface, and an axial lumen defined by the inner surface and extending between the proximal end and the distal end. The coupler further comprises a distal portion comprising a flange surrounding the distal end of the tubular portion.

In another embodiment, the proximal end of the coupler comprises a circumferential lip.

In one embodiment, the flange is generally elliptical in shape. In a preferred embodiment, the flange comprises a dorsal surface, and the flange further comprises one or more than one fin connecting the dorsal surface of the flange to the outer surface of the tubular portion. In a particularly preferred embodiment, the one or more than one fin comprises a front fin and a back fin.

In another embodiment, the tubular portion comprises one or more than one barb extending from the inner surface into the axial lumen and directed distally. In a preferred embodiment, the flange comprises a circumferential inner edge bordering the distal end of the tubular portion, and where the circumferential inner edge creates a circumferential ledge extending into the axial lumen. In another preferred embodiment, the tubular portion comprises one or more than one notch extending into the inner surface of the tubular portion.

In one embodiment, the coupler further comprises one or more than one substance embedded in the coupler that inhibits inflamation and smooth muscle proliferation. In another embodiment, the coupler further comprises sirolimus embedded in the coupler.

According to another embodiment of the present invention, there is provided a coupler for creating an anastomosis between a first vessel and a second vessel. The coupler comprises a proximal portion comprising a tubular portion having a proximal end, a distal end, an outer surface, an inner surface, and an axial lumen defined by the inner surface and extending between the proximal end and the distal end. The coupler further comprises a distal portion comprising a flange surrounding the distal end of the tubular portion. The flange comprises a dorsal surface and the flange further comprises one or more than one fin connecting the dorsal surface of the flange to the outer surface of the tubular portion.

According to another embodiment of the present invention, there is provided a coupling vessel for creating an anastomosis between a first vessel and a second vessel. The coupling vessel comprises a proximal portion comprising a tubular portion having a proximal end, a distal end, an outer surface, an inner surface, and an axial lumen defined by the inner surface and extending between the proximal end and the distal end. The coupling vessel further comprises a distal portion comprising a flange surrounding the distal end of the tubular portion.

In one embodiment, the proximal end of the coupling vessel comprises a circumferential lip.

In one embodiment, the flange is generally elliptical in shape. In a preferred embodiment, the flange comprises a dorsal surface and where the flange further comprises one or more than one fin connecting the dorsal surface of the flange to the outer surface of the tubular portion. In a particularly preferred embodiment, the one or more than one fin comprises a front fin and a back fin.

In one embodiment, the coupling vessel further comprises one or more than one substance embedded in the coupling vessel that inhibits inflamation and smooth muscle proliferation. In another embodiment, the coupling vessel further comprises sirolimus embedded in the coupling vessel.

In another embodiment, the coupling vessel comprises a second flange surrounding the proximal end of the tubular portion.

According to another embodiment of the present invention, there is provided a coupling vessel for creating an anastomosis between a first vessel and a second vessel. The coupling vessel comprises a proximal portion comprising a tubular portion having a proximal end, a distal end, an outer surface, an inner surface, and an axial lumen defined by the inner surface and extending between the proximal end and the distal end. The coupling vessel further comprises a distal portion comprising a flange surrounding the distal end of the tubular portion. The flange comprises a dorsal surface and the flange further comprises one or more than one fin connecting the dorsal surface of the flange to the outer surface of the tubular portion.

According to another embodiment of the present invention, there is provided a method for creating an anastomosis between a first vessel and a second vessel. The method comprises a) selecting the first vessel and the second vessel; b) providing a coupler according to the present invention; and c) joining the first vessel to the coupler through the proximal end of the tubular portion and joining the second vessel to the coupler through the distal portion, thereby creating an anastomosis with fluid continuity between the first vessel and the second vessel. Joining the second vessel to the coupler comprises creating an opening in the second vessel and passing the flange into the opening.

In one embodiment of the method, the tubular portion further comprises one or more than one barb extending from the inner surface into the axial lumen and directed distally, and joining the first vessel to the coupler comprises engaging the one or more than one barb on the first vessel.

In another embodiment of the method, the tubular portion further comprises one or more than one notch extending into the inner surface of the tubular portion, and joining the first vessel to the coupler comprises engaging the one or more than one notch on the first vessel with the first vessel.

In one embodiment of the method, the flange comprises a circumferential inner edge bordering the distal end of the tubular portion, and the circumferential inner edge creates a circumferential ledge extending into the axial lumen. In this embodiment, joining the first vessel to the coupler comprises advancing the first vessel into the coupler until the first vessel encounters the circumferential ledge.

In a preferred embodiment of the method, the method additionally comprises joining the first vessel to a third vessel using a second coupler, thereby creating an anastomosis with fluid continuity between the third vessel and the second vessel through the first vessel.

According to another embodiment of the present invention, there is provided a method for creating an anastomosis between a first vessel and a second vessel. The method comprises a) selecting the first vessel and the second vessel; b) providing a coupling vessel according to the present invention; and c) joining the first vessel to the coupling vessel through the proximal end of the tubular portion and joining the second vessel to the coupling vessel through the distal portion, thereby creating an anastomosis with fluid continuity between the first vessel and the second vessel. Joining the second vessel to the coupling vessel comprises creating an opening in the second vessel and passing the flange into the opening.

In one embodiment of either of the methods, the first vessel is selected from the group consisting of a prosthetic blood vessel, an autologous blood vessel, a heterologous blood vessel harvested in vivo, a cadaveric blood vessel, and a blood vessel created from a plurality of the foregoing; and the second vessel is a blood vessel. In a preferred embodiment, the first vessel is an aorta, and where the second vessel is a coronary artery. In another preferred embodiment, the first vessel or the second vessel or both are selected from the group consisting of a bile duct and a lymphatic vessel.

In one embodiment of either of the methods, the first vessel has an exterior surface, and joining the first vessel to the coupling vessel comprises applying an adhesive to the exterior surface of the first vessel, the inner surface of the coupling vessel, or both the exterior surface of the first vessel and the inner surface of the coupling vessel. In a preferred embodiment of either of the methods, the adhesive comprises a cyanoacrylate.

In one embodiment of either of the methods, joining the first vessel to the coupling vessel comprises suturing or clipping the proximal end of the tubular portion to the first vessel.

In another embodiment of either of the methods, the flange comprises a dorsal surface, and the flange further comprises one or more than one fin connecting the dorsal surface of the flange to the outer surface of the tubular portion, and joining the second vessel to the coupling vessel further comprises attaching the one or more than one fin to the second vessel.

In a preferred embodiment of either of the methods, the flange comprises a dorsal surface, and joining the second vessel to the coupler further comprises applying an adhesive to the dorsal surface of the flange such that contact of the dorsal surface of the flange causes adhesion between the coupler and the second vessel. In a particularly preferred embodiment of either of the methods, the adhesive comprises a cyanoacrylate.

In another embodiment of either of the methods, the proximal end of the tubular portion comprises a circumferential lip, and joining the first vessel to the coupler and joining the second vessel to the coupler comprises grasping the circumferential lip of the tubular portion.

In a preferred embodiment, the coupling vessel comprises a second flange surrounding the proximal end of the tubular portion, and joining the first vessel to the coupling vessel comprises creating an opening in the first vessel and passing the second flange into the opening in the first vessel.

According to another embodiment of the present invention, there is provided means for creating an anastomosis between a first vessel and a second vessel. According to another embodiment of the present invention, there is provided a method for creating an anastomosis between a first vessel and a second vessel. The method comprises a) selecting the first vessel and the second vessel; b) providing the means of the present invention; and c) joining the first vessel to the coupler through the proximal end of the tubular portion and joining the second vessel to the coupler through the distal portion, thereby creating an anastomosis with fluid continuity between the first vessel and the second vessel.

### FIGURES

These and other features, aspects and advantages of the present invention will become better understood with regard to the following description, appended claims, and accompanying figures where:

FIG. 1 is a lateral perspective view of a coupler according to the present invention;

FIG. 2 is a lateral perspective cutaway view of a coupler as shown in FIG. 1;

FIG. 3 is a top perspective view of a coupler as shown in FIG. 1;

FIG. 4 is another top perspective view of a coupler as shown in FIG. 1, showing additional detail in relief;

FIG. 5 is a partial, lateral perspective view of a coupling vessel according to the present invention;

FIG. 6 is a partial, lateral perspective cutaway view of a coupling vessel as shown in FIG. 5;

FIG. 7 is a partial, top perspective view of a coupling vessel as shown in FIG. 5;

FIG. 8 is another partial, top perspective view of a coupling vessel as shown in FIG. 5, showing additional detail in relief;

FIG. 9 is a lateral perspective cutaway view of an anastomosis between a first vessel and a second vessel using a coupler according to the present invention;

FIG. 10 is an axial perspective cutaway view of an anastomosis between a first vessel and a second vessel using a coupler according to the present invention;

FIG. 11 is a lateral perspective cutaway view of an anastomosis between a first vessel and a second vessel using a coupling vessel according to the present invention; and

FIG. 12 is an axial perspective cutaway view of an anastomosis between a first vessel and a second vessel using a coupling vessel according to the present invention.

### DESCRIPTION

According to one embodiment of the present invention, there is provided a coupler suitable for creating an anastomosis between a first vessel and a second vessel. According to another embodiment of the present invention, there is provided a coupling vessel suitable for creating an anastomosis between a first vessel and a second vessel. According to another embodiment of the present invention, there is provided a method for creating anastomoses between a first vessel and a second vessel using a coupler according to the present invention to attach the first vessel to the second vessel. According to another embodiment of the present invention, there is provided a method for creating anastomoses between a first vessel and a second vessel using a coupling vessel according to the present invention to attach the first vessel to the second vessel. Among other uses, the devices and methods of the present invention allow the formation of anastomoses between a first blood vessel and a second blood vessel, where the second blood vessel has a diameter too small for creating an anastomosis with the first blood vessel by traditional suturing techniques. The devices and methods of the present invention will now be disclosed in more detail.

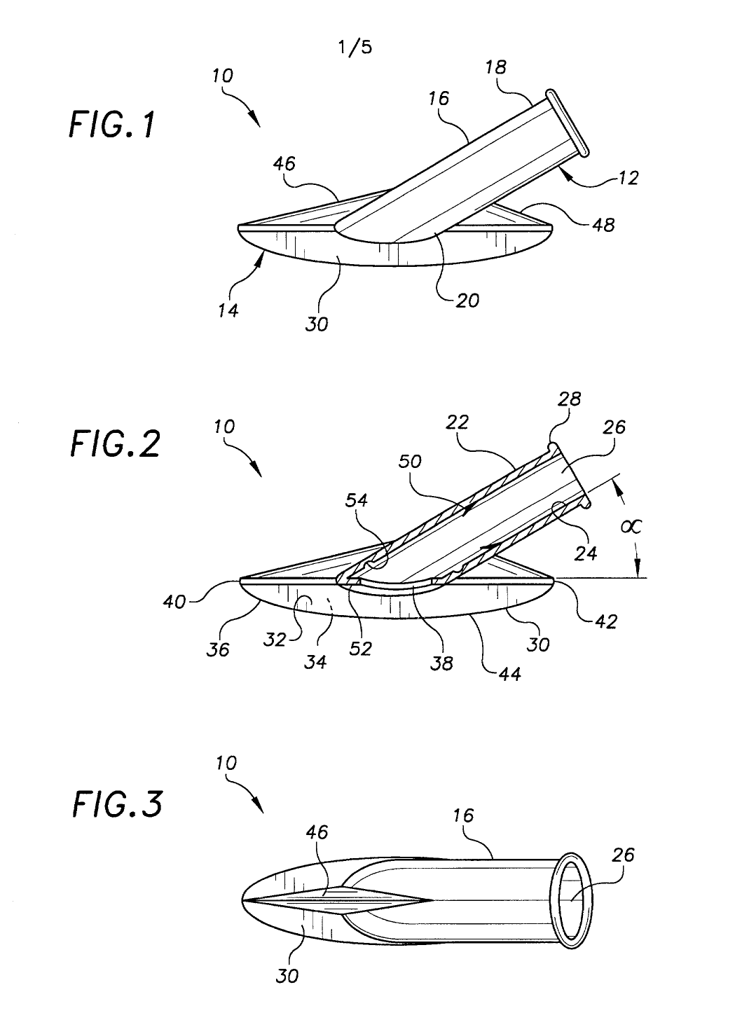

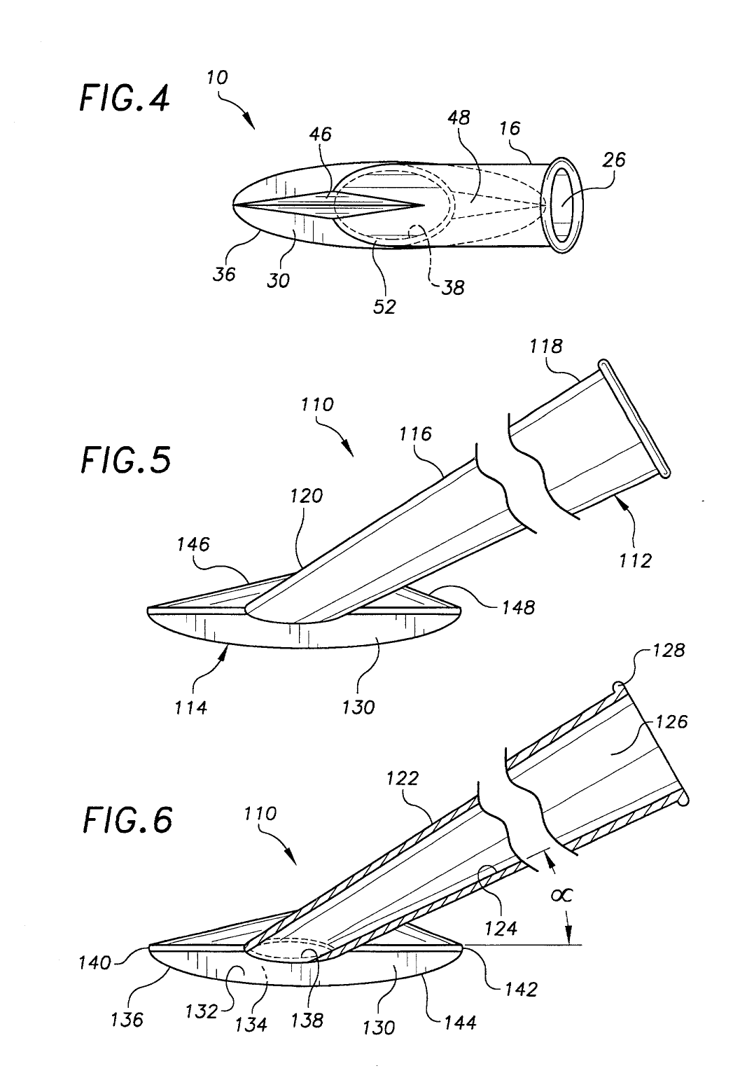

In one embodiment, the present invention is a coupler suitable for creating an anastomosis between a first vessel and a second vessel. Referring now to FIG. 1, FIG. 2, FIG. 3 and FIG. 4, there are shown, respectively, a lateral perspective view; a lateral perspective cutaway view; and two top perspective views of a coupler according to the present invention. As can be seen, the coupler 10 comprises a proximal portion 12 and a distal portion 14. The proximal portion 12 comprises a tubular portion 16 having a proximal end 18, a distal end 20, an outer surface 22, an inner surface 24, and an axial lumen 26 defined by the inner surface 24 and extending between the proximal end 18 and the distal end 20. In a preferred embodiment, the proximal end 18 comprises a circumferential lip 28.

The distal portion 14 comprises a flange 30 surrounding the distal end 20 of the tubular portion 16. In a preferred embodiment, the flange 30 is generally elliptical, as shown. However, the flange 30 can also be other shapes such as round or diamond shaped, as will be understood by those with skill in the art with reference to this disclosure. The flange 30 comprises a dorsal surface 32, a ventral surface 34, a circumferential outer edge 36 and a circumferential inner edge 38. The flange 30 has a front end 40, a back end 42 and two lateral edges 44 between the front end 40 and the back end 42.

Preferably, the axis of the tubular portion 16 makes an angle a with the plane of the outer edge 36 that is preset according to the intended use of the coupler 10. For example, when the coupler 10 is to be used to join a blood vessel, such as an internal mammary artery, to a coronary artery, the angle α is preferably about 30°, as shown, though other angles will be appropriate depending on the intended use for the coupler 10, as will be understood by those with skill in the art with reference to this disclosure.

In a preferred embodiment, the flange 30 further comprises one or more than one fin connecting the dorsal surface 32 of the flange 30 to the outer surface 22 of the tubular portion 16. In a preferred embodiment, as shown, the flange 30 comprises two fins, a front fin 46 and a back fin 48. However, the number and location of the fins will vary depending on the intended use of the coupler 10 as will be understood by those with skill in the art with reference to this disclosure. The fin or fins, when present, assist in stabilizing the angle α between the tubular portion 16 and the flange 30, and assist in joining the coupler 10 to the second vessel, as will be discussed in greater detail below.

In another preferred embodiment, the tubular portion 16 further comprises one or more than one barb 50 extending from the inner surface 24 into the axial lumen 26 and directed distally. In a preferred embodiment, the tubular portion comprises two barbs. Preferably, each barb 50 is present about midway through the tubular portion 16. However, the one or more than one barb 50 can be any number of barbs and the one or more than one barb 50 can be present at any suitable location within the tubular portion 16, as will be understood by those with skill in the art with reference to this disclosure.

In another preferred embodiment, as shown, the circumferential inner edge 38 creates a circumferential ledge 52 extending into the axial lumen 26. The ledge 52, when present, functions to stop distal translation of a first vessel in the axial lumen 26, and allows an operator to determine through tactile sensation when the first vessel in inserted completely through the tubular lumen 26, among other functions as disclosed in this disclosure.

In another preferred embodiment, as shown, the tubular portion 16 comprises one or more than one notch 54 extending into the inner surface 24 of the tubular portion 16. The one or more than one notch 54, when present assists in holding a first vessel by grasping the adventitia of the first vessel in the tubular portion 16.

The coupler 10 can comprise either natural material or synthetic material, or a combination of a natural material and a synthetic material. When the coupler 10 is going to be used to join a first vessel with a second vessel in vivo, the coupler 10 preferably comprises a biocompatible material that is resistant to in vivo degradation, such as polytetrafluorethylene, polyethylene terephtalate or a polyurethane composite. The tubular portion 16 should have a compliance substantially matching the second vessel. When present, the one or more than one fin 46, 48 should be stiffer than the tubular portion 16 to allow manipulation of the coupler 10 by grasping the one or more than one fin 46, 48 with an appropriate instrument. The flange 30 should be less stiff than the tubular portion 16 to allow the flange 30 to fold easily and be inserted into the second vessel.

Additionally, the coupler 10 can have one or more than one substance embedded in the coupler 10 that inhibits inflamation and smooth muscle proliferation and, thereby, decreases the incidence of restenosis at the site of the anastomosis. In one embodiment, the substance is sirolimus (rapamycin; Rapamune® Wyeth), a macrolide antibiotic. Further, the coupler 10 is preferably nonthrombogenic, such as by being coated with a substance to prevent platelet aggregation, such as dipyridamole.

The dimensions of the coupler 10 will depend on its intended use, as will be understood by those with skill in the art with reference to this disclosure. For example, when used for anastomosing a saphenous vein graft to a coronary artery, the tubular portion 16 of the coupler 10 would be about 100 μm thick with a circumferential lip 28 about 200 μm thick. The diameter of the axial lumen 26 at the proximal end 18 would be about 3 mm. The axial length of the tubular portion 16 would be between about 4 mm and about 6 mm. The shortest length between the two lateral edges 44 of the flange 30 would be between about 2.5 mm and about 3.5 mm, and the longest length between the front edge and the back edge of the flange 30 would be between about 3 mm and about 5 mm. These dimensions are exemplary only.

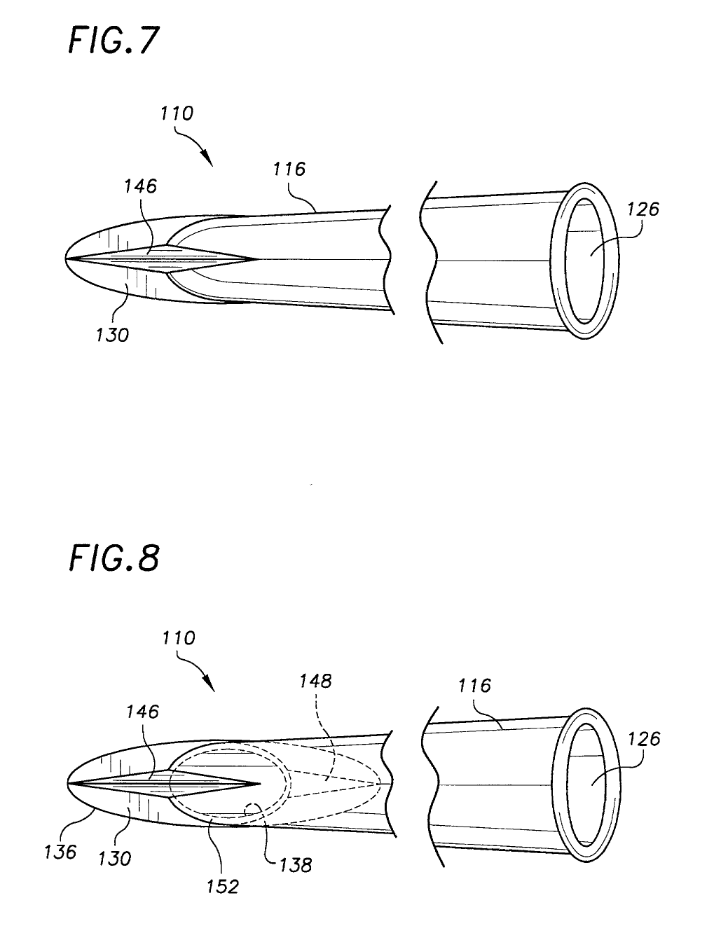

According to another embodiment, the present invention is a coupling vessel suitable for creating an anastomosis between a first vessel and a second vessel. Referring now to FIG. 5, FIG. 6, FIG. 7 and FIG. 8, there are shown, respectively, a partial, lateral perspective view; a partial, lateral perspective cutaway view; and two partial, top perspective views of a coupling vessel according to the present invention. As can be seen, the coupling vessel 110 comprises a proximal portion 112 and a distal portion 114. The proximal portion 112 comprises an elongated tubular portion 116 having a proximal end 118, a distal end 120, an outer surface 122, an inner surface 124, and an axial lumen 126 defined by the inner surface 124 and extending between the proximal end 118 and the distal end 120. In a preferred embodiment, the proximal end 118 comprises a circumferential lip 128.

The distal portion 114 comprises a flange 130 surrounding the distal end 120 of the tubular portion 116. In a preferred embodiment, the flange 130 is generally elliptical, as shown. However, the flange 130 can also be other shapes such as round or diamond shaped, as will be understood by those with skill in the art with reference to this disclosure. The flange 130 comprises a dorsal surface 132, a ventral surface 134, a circumferential outer edge 136 and a circumferential inner edge 138. The outer edge 136 has a front end 140, a back end 142 and two lateral edges 144 between the front end 140 and the back end 142.

Preferably, the axis of the tubular portion 116 makes an angle a with the plane of the outer edge 136 that is preset according to the intended use of the coupling vessel 110. For example, when the coupling vessel 110 is to be used to join a blood vessel, such as an internal mammary artery, to a coronary artery, the angle α is preferably about 30°, as shown, though other angles could be appropriate depending on the intended use for the coupling vessel 110, as will be understood by those with skill in the art with reference to this disclosure.

In a preferred embodiment, the flange 130 further comprises one or more than one fin connecting the dorsal surface 132 of the flange 130 to the outer surface 122 of the tubular portion 116. In a preferred embodiment, as shown, the flange 130 comprises two fins, a front fin 146 and a back fin 148. However, the number and location of the fins will vary depending on the intended use of the coupling vessel 110 as will be understood by those with skill in the art with reference to this disclosure. The fin or fins, when present, assist in stabilizing the angle α between the tubular portion 116 and the flange 130, and assist in joining the coupling vessel 110 to the second vessel, as will be discussed in greater detail below.

The coupling vessel 110 can comprise either natural material or synthetic material, or a combination of a natural material and a synthetic material. When the coupling vessel 110 is going to be used to join a first vessel with a second vessel in vivo, the coupling vessel 110 preferably comprises a biocompatible material that is resistant to in vivo degradation, such as polytetrafluorethylene, polyethylene terephtalate or a polyurethane composite. The tubular portion 116 should have a compliance substantially matching the second vessel. When present, the one or more than one fin 146, 148 should be stiffer than the tubular portion 116 to allow manipulation of the coupling vessel 110 by grasping the one or more than one fin 146, 148 with an appropriate instrument. The flange 130 should be less stiff than the tubular portion 116 to allow the flange 130 to fold easily and be inserted into the second vessel.

Additionally, the coupling vessel 110 can have one or more than one substance embedded in the coupling vessel 110 that inhibits inflamation and smooth muscle proliferation and, thereby, decreases the incidence of restenosis at the site of the anastomosis. In one embodiment, the substance is sirolimus (rapamycin; Rapamune® Wyeth), a macrolide antibiotic. Further, the coupling vessel 110 is preferably nonthrombogenic, such as by being coated with a substance to prevent platelet aggregation, such as dipyridamole.

The dimensions of the coupling vessel 110 will depend on its intended use, as will be understood by those with skill in the art with reference to this disclosure. For example, when used for anastomosing the aorta to a coronary artery, the tubular portion 116 of the coupling vessel 110 would be between about 100 μm thick and about 300 μm thick. The diameter of the axial lumen 126 at the proximal end 118 would be about 4 mm at the proximal end 118 (to be joined to the aorta) and about 3 mm at the distal end 120 (to be joined to the coronary artery). The axial length of the tubular portion 116 would be between about 10 cm and about 20 cm. The shortest length between the two lateral edges 144 would be between about 2.5 mm and about 3.5 mm, and the longest length between the front edge and the back edge would be between about 3 mm and about 5 mm. These dimensions are exemplary only.

Additionally, the coupling vessel 110 can comprise a second flange (not shown) at the proximal end 112 having equivalent elements to the first flange 130 at its distal end 114, except that the size of the second flange can be proportionally larger, as will be understood by those with skill in the art with reference to this disclosure.

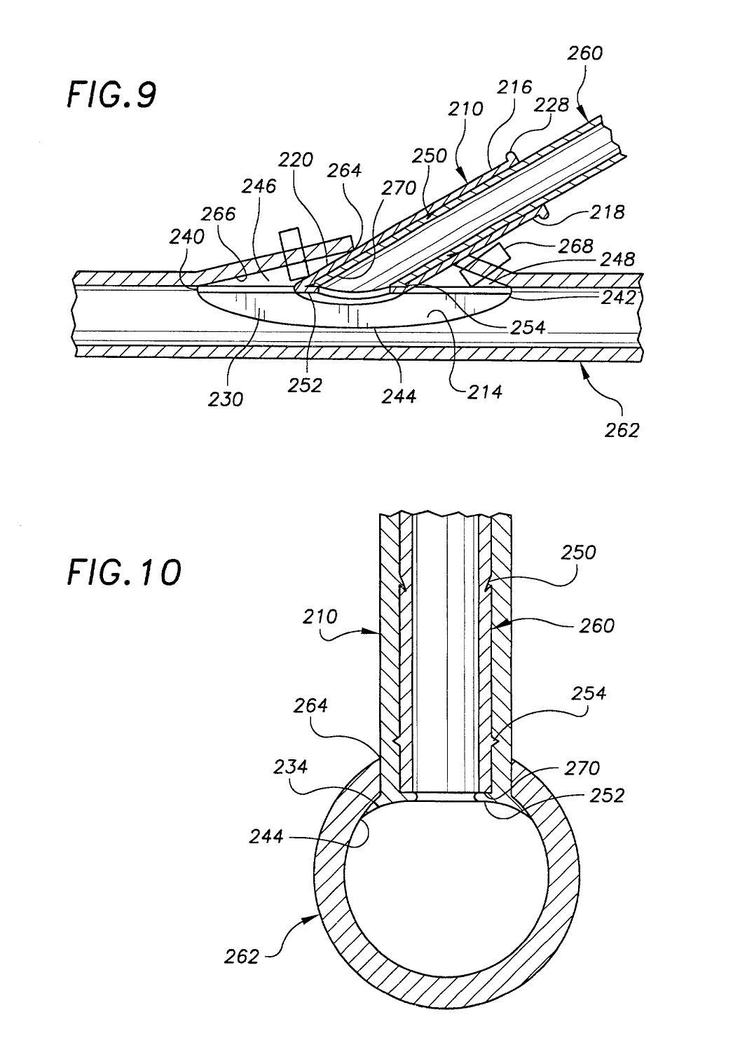

According to another embodiment of the present invention, there is provided a method for creating an anastomosis between a first vessel and a second vessel using a coupler according to the present invention. Referring now to FIG. 9 and FIG. 10, there are shown, respectively, a lateral perspective cutaway view and an axial perspective cutaway view of an anastomosis between a first vessel 260 and a second vessel 262 using a coupler 210 according to the present invention. The method comprises, first selecting the first vessel 260 and the second vessel 262. For example, when the second vessel 262 is a blood vessel, the first vessel 260 can be a prosthetic blood vessel, an autologous blood vessel, a heterologous blood vessel harvested in vivo, a cadaveric blood vessel, a blood vessel created from a plurality of the foregoing, or another suitable first vessel 260. In a preferred embodiment, the first vessel 260 is an autologous blood vessel, such as a saphenous vein or an internal mammary artery, and the second vessel 262 is a coronary artery.

Next, a suitable coupler 210 according to the present invention is provided. Then, the first vessel 260 is joined to the coupler 210 through the proximal end 218 of the tubular portion 216, and the second vessel 262 is joined to the coupler 210 through the distal portion 214, thereby creating an anastomosis with fluid continuity between the first vessel 260 and the second vessel 262. Though this method is disclosed below with reference to initially joining the coupler 210 to the first vessel 260, the coupler 210 can be joined to the second vessel 262 before joining the coupler 210 to the first vessel 260, as will be understood by those with skill in the art with reference to this disclosure.

The first vessel 260 is joined to the coupler 210 as follows. The distal end 270 of the first vessel 260 is inserted into the proximal end 218 of the axial lumen 226 of the tubular portion 216 of the coupler 210 and advanced distally. Preferably, the distal end 270 of the first vessel 260 is advanced until it covers the inner surface 224 of the tubular portion 216. The first vessel 260 is affixed to the coupler 210 using one or more techniques. For example, the first vessel 260 can be affixed to the coupler 210 by applying a suitable adhesive to the exterior surface of the first vessel 260, the inner surface 224 of the tubular portion 216 or both. Suitable adhesives are the same as disclosed below for affixing the coupler 210 to the second vessel 262. Additionally, when present, the one or more than one barb 250 on the inner surface 224 of the tubular portion 216 engages the adventitia of the first vessel 260 thereby preventing translocation of the first vessel 260 proximally. Further, when the method is being used to create an anastomosis between two blood vessels, the one or more than one notch 254, when present, assists in holding the first vessel 260 in the coupler 210 by grasping the adventitia of the first blood vessel. This is accomplished by slightly flexing and releasing the distal end 220 of the tubular portion 216 causing the one or more than one notch 254 to open and close. Additionally, the proximal end 218 of the tubular portion 216 can be sutured or clipped to the first vessel 260.

Next, the coupler 210 is joined to the second vessel 262 as follows. First, an opening 264 is made in the second vessel 262. Preferably, the opening 264 is parallel to the long axis of the second vessel 262. The length of the opening 264 is sufficient to permit entry of the flange 230 on the coupler 210 and to permit entry of the distal end 220 of the tubular portion 216 of the coupler 210. Then, the lateral edges 244 of the flange 230 are compressed toward the front to back axis of the flange 230 and the front end 240 of the flange 230 is inserted into the opening 264. Next, the back end 242 of the flange 230 is inserted into the opening 264 and the coupler 210 is pulled slightly away from the second vessel 262 in order to cause contact of the dorsal surface 232 of the flange 230 with the inner surface 266 of the second vessel 262. If present, the one or more than one fin 246, 248 on the dorsal surface 232 cause the edges of the opening 264 to evert slightly around the one or more than one fin 246, 248.

The coupler 210 is affixed to the second vessel 262 using one or more techniques. For example, the coupler 210 can be affixed to the second vessel 262 by applying a suitable adhesive to the dorsal surface 232 of the flange 230 such that contact of the dorsal surface 232 of the flange 230 causes adhesion between the coupler 210 and the second vessel 262. Preferably, the adhesive is applied before introducing the flange 230 into the opening 264. Suitable adhesives are selected based upon the composition of the coupler 210 and the composition of the second vessel 262, as will be understood by those with skill in the art with reference to this disclosure. For example, where the coupler 210 comprises polyurethane and the second vessel 262 is a in situ blood vessel, a suitable adhesive would be a cyanoacrylate. In a preferred embodiment, the adhesive is 2-octylcyanoacrylate from (sold under the name Dermabond®, from Johnson & Johnson, US). The coupler 210 can also be affixed to the second vessel 262, for example, by attaching the everted edges of the opening 264 to the fins 246, 248 using a suitable clip 268, as shown, or by passing suture through the everted edges of the opening 264 and fin 246, 248. Once the first vessel 260 and the second vessel 262 are joined to the coupler 210, the anastomosis is complete.

When present, the ledge 252 created by the circumferential inner edge 238 of the flange 230 functions to stop distal translation of the first vessel 260 in the axial lumen 226, and allows an operator to determine through tactile sensation when the first vessel 260 in inserted completely through the tubular lumen. Further, when the first vessel 260 is a blood vessel, the ledge 252 covers the cut distal end of the blood vessel, thereby preventing exposure of the cut distal end to blood products when blood flow occurs between the first vessel 260 and the second vessel 262, reducing the incidence of clotting and proliferation at the juncture of the first vessel 260 and the second vessel 262, and allowing endothelialization over the ventral surface of the flange 234 to join the endothelial surface of the first vessel. Additionally, when present, the operator uses the lip 228 to assist in grasping the coupler 210 during manipulation of the coupler 210.

Additionally, the method can comprise creating an anastomosis between a first vessel and a second vessel using a coupler according to the present invention as disclosed above, and creating an anastomosis between the first vessel and a third vessel using another coupler according to the present invention and the techniques disclosed in this disclosure. For example, a coupler according to the present invention can be used to join the distal end of a saphenous vein to a coronary artery, and another coupler can be used to join the proximal end of the saphenous vein to the aorta, thereby establishing fluid communication between the aorta and the coronary artery though the saphenous vein.

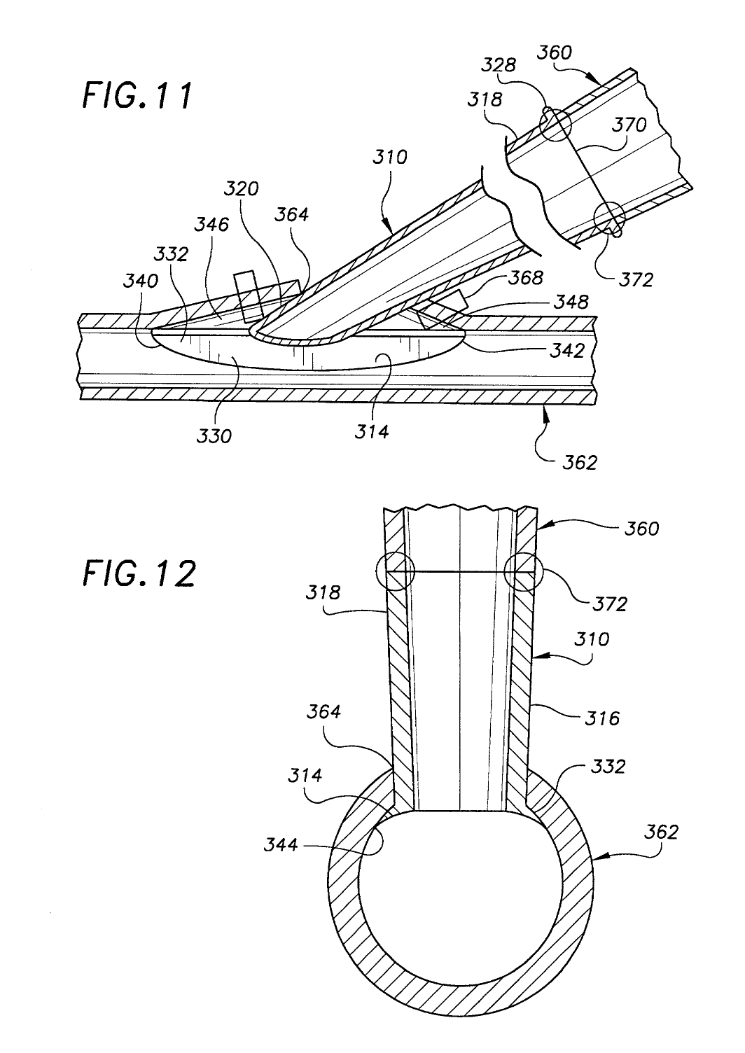

According to another embodiment of the present invention, there is provided another method for creating an anastomosis between a first vessel 360 and a second vessel 362 using a coupling vessel 310 according to the present invention. Referring now to FIG. 11 and FIG. 12, there are shown, respectively, a lateral perspective cutaway view and an axial perspective cutaway view of an anastomosis between a first vessel 360 and a second vessel 362 using a coupling vessel 310 according to the present invention. The method comprises, first selecting the first vessel 360 and second vessel 362. For example, when the second vessel 362 is a blood vessel, the first vessel 360 can be a prosthetic blood vessel, an autologous blood vessel, a heterologous blood vessel harvested in vivo, a cadaveric blood vessel, a blood vessel created from a plurality of the foregoing, or another suitable first vessel 360. In a preferred embodiment, the first vessel 360 is an autologous blood vessel, such as the aorta, and the second vessel 362 is a coronary artery.

Next, a suitable coupling vessel 310 according to the present invention is provided. Then, the first vessel 360 is joined to the coupling vessel 310 at the proximal end 318 of the tubular portion 316 and the coupling vessel 310, and the second vessel 362 to the coupling vessel 310 through the distal portion 314 of the coupling vessel 310, thereby creating an anastomosis between the first vessel 360 and the second vessel 362. Though shown as an end to end anastomosis, the first vessel 360 can also be joined to the coupling vessel 310 by any suitable configuration, such as a side to end anastomosis, as will be understood by those with skill in the art with reference to this disclosure. Additionally, though shown as an abutting end to end anastomosis, the first vessel 360 can also be joined to the coupling vessel 310 by having the one end of the first vessel pass partially or fully into the proximal end 318 of the tubular portion 316, as will be understood by those with skill in the art with reference to this disclosure. Though this method is disclosed below with reference to initially joining the coupling vessel 310 to the first vessel 360, the coupling vessel 310 can be joined to the second vessel 362 before joining the coupling vessel 310 to the first vessel 360, as will be understood by those with skill in the art with reference to this disclosure.

The first vessel 360 is joined to the coupling vessel 310 by creating an opening 370 in the first vessel 360, such as creating vasculotomy or cutting one end of a blood vessel. Then, the proximal end 318 of the tubular portion 316 of the coupling vessel 310 is connected to the edge of the opening 370 in the first vessel 360 or around the edges of the opening 370 in the first vessel 360, using techniques appropriate for the material and size of the coupling vessel 310 and the first vessel 360, as will be understood by those with skill in the art with reference to this disclosure. For example, where the first vessel 360 is a blood vessel and the coupling vessel 310 is a prosthetic vascular graft, the coupling vessel 310 and first vessel 360 can be joined using sutures 372, as shown, or using surgical clips. Further, the method can comprise creating an anastomosis between a first vessel and a second vessel using a coupling vessel comprising a flange at both the proximal end 312 (not shown) and the distal end 314 (as shown) according to the present invention as disclosed above. When two flanges are present on the coupling vessel 310, the anastomosis between the first vessel 370 and the coupling vessel 310 is created by passing the flange at the proximal end 312 into an opening made in the first vessel 370 and joining the flange at the proximal end 312 using the same techniques as disclosed for joining the flange at the distal end 314.

Next, the coupling vessel 310 is joined to the second vessel 362 as follows. First, an opening 364 is made in the second vessel 362. Preferably, the opening 364 is parallel to the long axis of the second vessel 362. The length of the opening 364 is sufficient to permit entry of the flange 330 on the coupling vessel 310 and to permit entry of the distal end 320 of the tubular portion 316 of the coupling vessel 310. Then, the lateral edges 344 of the flange 330 are compressed toward the front to back axis of the flange 330 and the front end 340 of the flange 330 is inserted into the opening 364. Next, the back end 342 of the flange 330 is inserted into the opening 364 and the coupling vessel 310 is pulled slightly away from the second vessel 362 in order to cause contact of the dorsal surface 332 of the flange 330 with the inner surface 366 of the second vessel 362. If present, the one or more than one fin 346, 348 on the dorsal surface 332 cause the edges of the opening 364 to evert slightly around the one or more than one fin 346, 348.

The coupling vessel 310 is affixed to the second vessel 362 using one or more techniques. For example, the coupling vessel 310 can be affixed to the second vessel 362 by applying a suitable adhesive to the dorsal surface 332 of the flange 330 such that contact of the dorsal surface 332 of the flange 330 causes adhesion between the coupling vessel 310 and the second vessel 362. Preferably, the adhesive is applied before introducing the flange 330 into the opening 364. Suitable adhesives are selected based upon the composition of the coupling vessel 310 and the composition of the second vessel 362, as will be understood by those with skill in the art with reference to this disclosure. For example, where the coupling vessel 310 comprises polyurethane and the second vessel 362 is a in situ blood vessel, a suitable adhesive would be a cyanoacrylate. In a preferred embodiment, the adhesive is 2-octylcyanoacrylate (sold under the name Dermabond®, from Johnson & Johnson, US). The coupling vessel 310 can also be affixed to the second vessel 362, for example, by attaching the everted edges of the opening 364 to the fin 346, 348 using a suitable clip 368, as shown, or by passing suture through the everted edges of the opening 364 and fin 346, 348. Once the first vessel 360 and the second vessel 362 are joined to the coupling vessel 310, the anastomosis is complete.

Additionally, when present, the operator uses the lip 328 at the proximal end of the coupling vessel 360 to grasp the coupling vessel 360 during manipulation of the coupling vessel 360.

Though the methods and devices of the present invention are disclosed specifically with respect to microvascular anastomoses, the devices and methods of the present invention can also be used to join a variety of hollow vessels having a variety of lumen sizes, as will be understood by those with skill in the art with reference to this disclosure. For example, the devices and methods of the present invention can be used to perform macrovascular anastomoses, bile duct anastomoses and lymphatic anastomoses. Additionally, the devices and methods of the present invention can be used to join hollow vessels that are not living tissues, particularly where the hollow vessels are small or delicate.

Therefore, although the present invention has been discussed in considerable detail with reference to certain preferred embodiments, other embodiments are possible, and the scope of the appended claims should not be limited to the description of preferred embodiments contained in this disclosure.

What is claimed is:

1. A coupler for creating an anastomosis between a first vessel and a second vessel, the coupler comprising:
a proximal portion comprising a tubular portion having a proximal end, a distal end, an outer surface, an inner surface, and an axial lumen defined by the inner surface and extending between the proximal end and the distal end; and
a distal portion comprising a flange surrounding the distal end of the tubular portion.

2. The coupler of claim 1, where the proximal end comprises a circumferential lip.

3. The coupler of claim 1, where the flange is generally elliptical in shape.

4. The coupler of claim 1, where the flange comprises a dorsal surface, and where the flange further comprises one or more than one fin connecting the dorsal surface of the flange to the outer surface of the tubular portion.

5. The coupler of claim 1, where the one or more than one fin comprises a front fin and a back fin.

6. The coupler of claim 1, where the tubular portion further comprises one or more than one barb extending from the inner surface into the axial lumen and directed distally.

7. The coupler of claim 1, where the flange comprises a circumferential inner edge bordering the distal end of the tubular portion; and
where the circumferential inner edge creates a circumferential ledge extending into the axial lumen.

8. The coupler of claim 1, where the tubular portion comprises one or more than one notch extending into the inner surface of the tubular portion.

9. The coupler of claim 1, further comprising one or more than one substance embedded in the coupler that inhibits inflamation and smooth muscle proliferation.

10. The coupler of claim 1, further comprising sirolimus embedded in the coupler.

11. A coupler for creating an anastomosis between a first vessel and a second vessel, the coupler comprising:
a proximal portion comprising a tubular portion having a proximal end, a distal end, an outer surface, an inner surface, and an axial lumen defined by the inner surface and extending between the proximal end and the distal end; and
a distal portion comprising a flange surrounding the distal end of the tubular portion;
where the flange comprises a dorsal surface; and
where the flange further comprises one or more than one fin connecting the dorsal surface of the flange to the outer surface of the tubular portion.

12. A coupling vessel for creating an anastomosis between a first vessel and a second vessel, the coupling vessel comprising:
a proximal portion comprising a tubular portion having a proximal end, a distal end, an outer surface, an inner surface, and an axial lumen defined by the inner surface and extending between the proximal end and the distal end; and
a distal portion comprising a first flange surrounding the distal end of the tubular portion.

13. The coupling vessel of claim 12, where the proximal end comprises a circumferential lip.

14. The coupling vessel of claim 12, where the first flange is generally elliptical in shape.

15. The coupling vessel of claim 12, where the first flange comprises a dorsal surface and where the first flange further comprises one or more than one fin connecting the dorsal surface of the first flange to the outer surface of the tubular portion.

16. The coupling vessel of claim 12, where the one or more than one fin comprises a front fin and a back fin.

17. The coupling vessel of claim 12, further comprising one or more than one substance embedded in the coupling vessel that inhibits inflamation and smooth muscle proliferation.

18. The coupling vessel of claim 12, further comprising sirolimus embedded in the coupling vessel.

19. The coupling vessel of claim 12, further comprising a second flange surrounding the proximal end of the tubular portion.

20. A coupling vessel for creating an anastomosis between a first vessel and a second vessel, the coupling vessel comprising:
a proximal portion comprising a tubular portion having a proximal end, a distal end, an outer surface, an inner surface, and an axial lumen defined by the inner surface and extending between the proximal end and the distal end; and
a distal portion comprising a flange surrounding the distal end of the tubular portion;
where the flange comprises a dorsal surface; and
where the flange further comprises one or more than one fin connecting the dorsal surface of the flange to the outer surface of the tubular portion.

21. A method for creating an anastomosis between a first vessel and a second vessel, the method comprising:
a) selecting the first vessel and the second vessel;
b) providing a first coupler according to claim 1; and
c) joining the first vessel to the first coupler through the proximal end of the tubular portion and joining the second vessel to the first coupler through the distal portion, thereby creating an anastomosis with fluid continuity between the first vessel and the second vessel; and
where joining the second vessel to the first coupler comprises creating an opening in the second vessel and passing the flange into the opening.

22. The method of claim 1, where the first vessel is selected from the group consisting of a prosthetic blood vessel, an autologous blood vessel, a heterologous blood vessel harvested in vivo, a cadaveric blood vessel, and a blood vessel created from a plurality of the foregoing; and
where the second vessel is a blood vessel.

23. The method of claim 1, where the first vessel is a saphenous vein or an internal mammary artery; and
where the second vessel is a coronary artery.

24. The method of claim 1, where the first vessel or the second vessel or both are selected from the group consisting of a bile duct and a lymphatic vessel.

25. The method of claim 1, where the first vessel has an exterior surface; and
where joining the first vessel to the first coupler comprises applying an adhesive to the exterior surface of the first vessel, the inner surface of the tubular portion, or both the exterior surface of the first vessel and the inner surface of the tubular portion.

26. The method of claim 25, where the adhesive comprises a cyanoacrylate.

27. The method of claim 1, where the tubular portion further comprises one or more than one barb extending from the inner surface into the axial lumen and directed distally; and
where joining the first vessel to the first coupler comprises engaging the one or more than one barb on the first vessel.

28. The method of claim 1, where the tubular portion further comprises one or more than one notch extending into the inner surface of the tubular portion; and
where joining the first vessel to the first coupler comprises engaging the one or more than one notch on the first vessel with the first vessel.

29. The method of claim 1, where joining the first vessel to the first coupler comprises suturing or clipping the proximal end of the tubular portion to the first vessel.

30. The method of claim 1, where the flange comprises a dorsal surface;
where the flange further comprises one or more than one fin connecting the dorsal surface of the flange to the outer surface of the tubular portion; and
where joining the first vessel to the first coupler comprises attaching the one or more than one fin to the second vessel.

31. The method of claim 1, where the flange comprises a dorsal surface; and
where joining the second vessel to the first coupler further comprises applying an adhesive to the dorsal surface of the flange such that contact of the dorsal surface of the flange causes adhesion between the first coupler and the second vessel.

32. The method of claim 31, where the adhesive comprises a cyanoacrylate.

33. The method of claim 1, where the flange comprises a circumferential inner edge bordering the distal end of the tubular portion;
where the circumferential inner edge creates a circumferential ledge extending into the axial lumen; and
where joining the first vessel to the first coupler comprises advancing the first vessel into the first coupler until the first vessel encounters the circumferential ledge.

34. The method of claim 1, where the proximal end of the tubular portion comprises a circumferential lip; and
where joining the first vessel to the first coupler and joining the second vessel to the first coupler comprises grasping the circumferential lip of the tubular portion.

35. The method of claim 1, additionally comprising joining the first vessel to a third vessel using a second coupler, thereby creating an anastomosis with fluid continuity between the third vessel and the second vessel through the first vessel.

36. A method for creating an anastomosis between a first vessel and a second vessel, the method comprising:
a) selecting the first vessel and the second vessel;
b) providing a coupling vessel according to claim 12; and
c) joining the first vessel to the coupling vessel through the proximal end of the tubular portion and joining the second vessel to the coupling vessel through the distal portion, thereby creating an anastomosis with fluid continuity between the first vessel and the second vessel; and
where joining the second vessel to the coupling vessel comprises creating an opening in the second vessel and passing the flange into the opening.

37. The method of claim 36, where the first vessel is selected from the group consisting of a prosthetic blood vessel, an autologous blood vessel, a heterologous blood vessel harvested in vivo, a cadaveric blood vessel, and a blood vessel created from a plurality of the foregoing; and
where the second vessel is a blood vessel.

38. The method of claim 36, where the first vessel is an aorta, and where the second vessel is a coronary artery.

39. The method of claim 36, where the first vessel or the second vessel or both are selected from the group consisting of a bile duct and a lymphatic vessel.

40. The method of claim 36, where the first vessel has an exterior surface; and
where joining the first vessel to the coupling vessel comprises applying an adhesive to the exterior surface of the first vessel, the inner surface of the coupling vessel, or both the exterior surface of the first vessel and the inner surface of the coupling vessel.

41. The method of claim 39, where the adhesive comprises a cyanoacrylate.

42. The method of claim 36, where joining the first vessel to the coupling vessel comprises suturing or clipping the proximal end of the tubular portion to the first vessel.

43. The method of claim 36, where the flange comprises a dorsal surface;
where the flange further comprises one or more than one fin connecting the dorsal surface of the flange to the outer surface of the tubular portion; and
where joining the second vessel to the coupling vessel further comprises attaching the one or more than one fin to the second vessel.

44. The method of claim 36, where the flange comprises a dorsal surface; and
where joining the second vessel to the coupling vessel further comprises applying an adhesive to the dorsal surface of the flange such that contact of the dorsal surface of the flange causes adhesion between the coupling vessel and the second vessel.

45. The method of claim 43, where the adhesive comprises a cyanoacrylate.

46. The method of claim 36, where the proximal end of the tubular portion comprises a circumferential lip; and
where joining the first vessel to the coupling vessel and joining the second vessel to the coupling vessel comprises grasping the circumferential lip of the tubular portion.

47. The method of claim 36, where the coupling vessel comprises a second flange surrounding the proximal end of the tubular portion; and
where joining the first vessel to the coupling vessel comprises creating an opening in the first vessel and passing the second flange into the opening in the first vessel.

47. Means for creating an anastomosis between a first vessel and a second vessel.

48. A method for creating an anastomosis between a first vessel and a second vessel, the method comprising:
a) selecting the first vessel and the second vessel;
b) providing the means of claim 46; and
c) joining the first vessel to the coupler through the proximal end of the tubular portion and joining the second vessel to the coupler through the distal portion, thereby creating an anastomosis with fluid continuity between the first vessel and the second vessel.
